(19) United States
(12) Patent Application Publication
(10) Pub. No.: US 2025/0288511 A1
(43) Pub. Date: Sep. 18, 2025

(54) OIL-IN-WATER EMULSION WITH A HIGH AMOUNT OF VITAMIN C INCLUDING A SILICON EMULSIFIER

(71) Applicant: L'OREAL, Paris (FR)

(72) Inventor: Audrey MONCUIT, Chevilly-Larue (FR)

(73) Assignee: L'OREAL, Paris (FR)

(21) Appl. No.: 18/862,620

(22) PCT Filed: May 9, 2023
(86) PCT No.: PCT/EP2023/062256

§ 371 Date: Nov. 4, 2024

(30) Foreign Application Priority Data
May 10, 2022 (FR) FR2204402

(51) Int. Cl. A61K 8/67; A61K 8/06; A61K 8/25; A61K 8/81; A61K 8/894; A61Q 19/00

(52) U.S. Cl. CPC A61K 8/676; A61K 8/062; A61K 8/25; A61K 8/8158; A61K 8/894; A61Q 19/00; A61K 2800/30

(57) ABSTRACT

The present invention relates to a composition in the form of an oil-in-water emulsion having a pH of between 4.5 and 6.5, comprising:
a) at least 5% by weight of ascorbic acid or one of the derivatives thereof with respect to the total weight of the composition;
b) at least one silicone emulsifier;
c) at least one polymer with a sulfonic group; and
d) at least one silicone elastomer;
   said composition being substantially free of cyclohexasiloxane.

It also relates to a method of cosmetic treatment of keratin materials, which comprises applying, on said keratin materials, a composition according to the invention, as well as the use of said composition in the cosmetic field, in particular for body or facial skin care.

The present invention relates to a composition in the form of an oil-in-water (O/V) emulsion having a pH of between 4.5 and 6.5, comprising:
a) at least 5% by weight of ascorbic acid or one of the derivatives thereof with respect to the total weight of the composition;
b) at least one silicone emulsifier;
c) at least one polymer with a sulfonic group;
d) at least one silicone elastomer; and
e) optionally at least one filler;
   said composition being substantially free of cyclohexasiloxane.

It also relates to a method of cosmetic treatment of keratin materials, which comprises applying, on said keratin materials, a composition according to the invention, as well as the use of said composition in the cosmetic field, in particular for body or facial skin care.

In the cosmetic field of care, cosmetic compositions making it possible to obtain both good sensory properties as well as a care effect are increasingly sought by users. Care effect denotes for example an effect countering the drying or aging of keratin materials and particularly the skin, this care effect being provided by active agents. As such, hydrophilic active agents, and particularly ascorbic acid (vitamin C), are found: this active agent is skin-regenerating thanks to the stimulation of collagen synthesis responsible for skin firmness, or depigmenting, as this vitamin reduces melanin production causing age spots. Ascorbic acid is also known for the antioxidant properties thereof.

However, this active agent is known to destabilize the medium wherein it is introduced, particularly by the acid function thereof. Furthermore, formulas containing a high amount (i.e., at least 5% by weight) of vitamin C are very tacky on the skin.

Adding silicones makes it possible to provide softness on application, as they provide a “bare skin” or “peachy skin” type skin finish. However, these compounds are frequently formulated with VOSiC (Volatile Organic Silicon Compounds), which are not environmentally friendly.

Consequently, there is a need for cosmetic compositions comprising an aqueous base, silicones and an effective quantity of ascorbic acid, which are stable, and which good sensory properties. In particular, there is a need for compositions having a “bare skin” or “peachy skin” type skin finish, and a fresh application.

Furthermore, there is a need for environmentally-friendly cosmetic compositions. Such compositions are particularly substantially free of volatile silicone(s); preservative(s), for example 2,6-di-tert-butyl 4-methylphenol (or butylhydroxytoluene or BHT); and/or chelating agent(s) for example EDTA.

The Applicant surprisingly observed that a cosmetic composition in the form of an O/W emulsion of given pH, comprising ascorbic acid or one of the derivatives thereof in an aqueous medium comprising specific silicones and optionally a filler, enables a finer and stabilizing dispersion of said silicones. Furthermore, such a composition is stable. These results are particularly obtained by adding a silicone emulsifier, which is an oil-in-water emulsifier.

The composition according to the invention typically has good sensory properties, such as a fresh and slick application and a non-tacky finish, as well as a viscosity typical of a serum. Finally, the composition according to the invention is more environmentally friendly, particularly as it substantially does not contain cyclohexasiloxane.

The present invention therefore relates to a composition, preferably cosmetic, in the form of an oil-in-water emulsion having a pH of between 4.5 and 6.5, comprising:
a) at least 5% by weight of ascorbic acid or one of the derivatives thereof with respect to the total weight of the composition;
b) at least one silicone emulsifier;
c) at least one polymer with a sulfonic group;
d) at least one silicone elastomer; and
e) optionally at least one filler;
   said composition being substantially free of cyclohexasiloxane.

The composition according to the invention is an oil-in-water emulsion. The term “oil-in-water emulsion” (or O/W emulsion) denotes a composition comprising an oily phase dispersed in an aqueous phase.

The composition according to the invention is stable. A composition is referred to as stable when the macroscopic appearance (clearness and homogeneity) does not change after at least 24 hours, preferably at least 1 month, preferably at least 2 months. In particular, the composition according to the invention has a clear and homogeneous appearance.

The composition according to the invention is preferably intended for a topical application, particularly on keratin materials, in particular the skin. It particularly comprises a physiologically acceptable medium, i.e., a medium compatible with all keratin materials, in particular the skin.

For the purposes of this invention, the term keratin materials denotes the skin and the appendages thereof. The term “skin” denotes facial skin and/or body skin, and the scalp. The term “appendages” denotes eyelashes, eyebrows, nails and hair, and particularly eyelashes and hair.

The present invention also relates to a method of cosmetic treatment of keratin materials, comprising application of a composition according to the invention on said keratin materials. Moreover, the invention also relates to the use of said composition in the cosmetic field, in particular for body or facial skin care.

### Ascorbic Acid and Derivatives Thereof

The composition according to the invention comprises ascorbic acid or one of the derivatives thereof, at a concentration of at least 5% by weight with respect to the total weight of the composition.

Preferably, the ascorbic acid or one of the derivatives thereof is present in the aqueous phase of the O/W emulsion.

The ascorbic acid according to the invention preferably corresponds to L-ascorbic acid, or vitamin C. It has a structure according to formula (II):

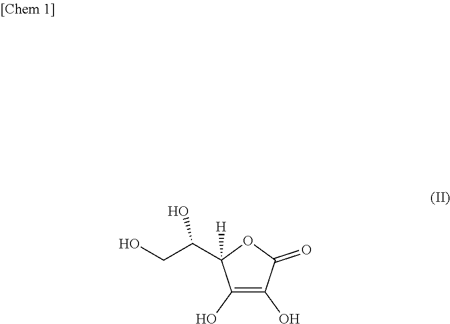 [SMILES: O=C1O[C@H]([C@@H](O)CO)C(O)=C1O]

The term “ascorbic acid derivative” preferably denotes a compound chosen from 5,6-di-O-dimethylsilylascorbate (particularly sold by Exsymol under the reference PRO-AA), DL-alpha-tocopheryl-DL-ascorbyl-phosphate potassium salt also known as Potassium Ascorbyl Tocopheryl Phosphate (sold by SEPPIC under the reference SEPIVITAL EPC), magnesium ascorbyl phosphate, sodium ascorbyl phosphate (sold by DSM under the reference Stay-C 50), disodium ascorbyl sulfate, sulfinyl-L-ascorbic acid, glucopyranosyl-L-ascorbic acid and ascorbyl glucoside.

The term “ascorbyl glucoside” denotes a condensation product of glucose, in D form, i.e., in a or p glucopyranose or a or p furanose, or in L form, with ascorbic acid, preferably in L form. Preferably, ascorbyl glucoside is L-ascorbic acid 2-O-α-D-glucopyranoside, particularly available from HAYASHIBARA.

Preferably, the composition according to the invention comprises ascorbic acid, preferably L-ascorbic acid.

The composition comprises at least 5% by weight of ascorbic acid or one of the derivatives thereof with respect to the total weight of the composition, preferably at least 7% by weight, more preferably at least 9% by weight, more preferably at least 10% by weight.

Preferably, the composition comprises from 5% to 20% by weight of ascorbic acid or one of the derivatives thereof, with respect to the total weight of the composition, more preferably from 7% to 15% by weight, even more preferably from 8% to 12% by weight.

### Silicone Emulsifier

The composition according to the invention comprises at least one silicone emulsifier.

The term “silicone emulsifier” denotes a silicone compound, preferably a polydimethylsiloxane, including at least one oxyalkylenated chain, particularly including at least one oxyethylenated (—OCH2CH2-) and/or oxypropylenated (—OCH2CH2CH2-) chain. The silicone emulsifier used in the present invention is soluble or dispersible in a monohydric alcohol such as ethanol.

By way of silicone emulsifier, mention can be made for example of (1) dimethicone copolyols such as those sold under the trade names DC 5329, DC 7439-146, DC 2-5695 by Dow Corning, bis-PEG/PPG-16/16 PEG/PPG-16/16 dimethicone such as that sold under the trade name Abil Care 85 by Evonik Goldschmidt, or dimethicone copolyol sold under the trade name ABIL EM 97 by Evonik Goldschmidt, and (2) alkyl-dimethicone copolyols such as laurylmethicone copolyol sold under the trade name Dow Corning 5200 Formulation Aid by Dow Corning, and cetyl dimethicone copolyol sold under the trade name ABIL EM 90 by Evonik Goldschmidt, or the polyglyceryl-4 isostearate/cetyl dimethicone copolyol/hexyllaurate mixture sold under the trade name ABIL WE 09 by Evonik Goldschmidt, and mixtures thereof.

Mention can for example be made of polydimethylsiloxane with an oxyethylene/oxypropylene termination sold in a mixture with caprylic/capric acid triglycerides under the trade name Abil Care 85 by Evonik Goldschmidt (INCI name: BIS-PEG/PPG-16/16 PEG/PPG-16/16 Dimethicone/Caprylic/Capric Triglyceride).

One or more co-emulsifiers can also be added to the silicone emulsifier. Advantageously, the co-emulsifier is chosen in the group comprising alkylated polyol esters. By way of example of alkylated polyol esters, particular mention can be made of glycerol and/or sorbitan esters and for example polyglycerol isostearate, such as the product sold under the trade name Isolan GI 34 by Evonik Goldschmidt, sorbitan isostearate, such as the product sold under the trade name Arlacel 987 by Croda, sorbitan and glycerol isostearate, such as the product sold under the trade name Arlacel 986 by Croda, and the mixtures thereof.

According to the embodiment of the composition according to the invention, the silicone emulsifier is a dimethicone copolyol, particularly bis-PEG/PPG-16/16 PEG/PPG-16/16 dimethicone. In particular, the silicone emulsifier is a mixture of bis-PEG/PPG-2 16/16 PEG/PPG-16/16 dimethicone and caprylic/capric triglycerides (87/13), such as that sold under the trade name Abil Care 85 by Evonik Goldschmidt.

The amount of silicone emulsifier (in active substance) ranges preferably from 0.1 to 5% by weight, preferably from 0.2 to 3% by weight, preferably from 0.3 to 1.5% by weight with respect to the total weight of the composition.

Polymer with Sulfonic Group

The composition according to the invention comprises at least one polymer with a sulfonic group.

The term “polymer with a sulfonic group” denotes a poly (2-acrylamido 2-methyl propane sulfonic) acid polymer, salts thereof, mixtures thereof, preferably a homo or a copolymer of a monomer with a sulfonic group, salts thereof, mixtures thereof.

Preferably, the polymer with a sulfonic group is present in the aqueous phase of the O/W emulsion.

These are water-soluble, water-dispersible or water-swellable polymers. The polymers used in accordance with the invention are homo- or copolymers capable of being obtained from at least one monomer with ethylenic unsaturation and with a sulfonic group, potentially in free or partially or totally neutralized form.

Preferably, the polymers with a sulfonic group used in accordance with the invention can be partially or completely neutralized by a mineral base (such as sodium hydroxide, potassium hydroxide or aqueous ammonia) or an organic base such as mono-, di- or triethanolamine, an aminomethyl propanediol, N-methylglucamine or basic amino acids such as arginine and lysine, or mixtures of these compounds. In general, they are neutralized. In the present invention, the term “neutralized” is intended to refer to polymers that have been neutralized completely or almost completely, that is to say neutralized to at least 90%.

The polymers with a sulfonic group used in the composition of the invention generally has a number-average molecular mass ranging from 1000 to 20,000,000 g/mol, preferably ranging from 20,000 to 5,000,000, and even more preferably from 100,000 to 1,500,000 g/mol.

The monomers with a sulfonic group of the polymer used in the composition of the invention are chosen particularly from vinylsulfonic acid, styrenesulfonic acid, (meth)acrylamido-(C1 C22)alkylsulfonic acids, N—(C1-C22)alkyl-(meth)acrylamido-(C1 C22)-alkylsulfonic acids such as undecyl-acrylamido-methane-sulfonic acid, as well as the partially or totally neutralized forms thereof, and mixtures thereof.

According to a preferred embodiment of the invention, the monomers with a sulfonic group are chosen from (meth)acrylamido(C1-C22)alkylsulfonic acids such as for example acrylamido-methane-sulfonic acid, acrylamido-ethane-sulfonic acid, acrylamido-propane-sulfonic acid, 2-acrylamido-2-methylpropane-sulfonic acid, 2-methacrylamido-2-methylpropane-sulfonic acid, 2-acrylamido-n-butane-sulfonic acid, 2-acrylamido-2,4,4-trimethylpentane-sulfonic acid, 2-methacrylamido-dodecyl-sulfonic acid, 2-acrylamido-2,6-dimethyl-3-heptane-sulfonic, as well as the partially or totally neutralized forms thereof, and mixtures thereof.

These polymers according to the invention can be crosslinked or non-crosslinked.

When the polymers are crosslinked, the crosslinking agents may be chosen from the olefinically-polyunsaturated compounds commonly used for crosslinking polymers obtained by radical polymerization.

As examples of crosslinking agents, mention can be made of divinylbenzene, diallyl ether, dipropylene glycol diallyl ether, polyglycol diallyl ethers, triethylene glycol divinyl ether, hydroquinone diallyl ether, di(meth)acrylate ethylene glycol, di(meth)acrylate tetraethylene glycol, trimethylol propane triacrylate, methylene-bis-acrylamide, methylene-bis-methacrylamide, triallylamine, triallyl cyanurate, diallyl maleate, tetraallylethylenediamine, tetra-allyloxyethane, trimethylolpropane diallyl ether, allyl (meth)acrylate, allyl ethers of alcohols as well as the allylic esters of phosphoric acid derivatives and/or vinylphosphonic acid derivatives, or mixtures of these compounds. According to a preferred embodiment of the invention, the crosslinking agent is chosen from methylene-bis-acrylamide, allyl methacrylate or trimethylol propane triacrylate (TMPTA).

The degree of crosslinking generally ranges from 0.01 to 10% in moles, and more particularly from 0.2 to 2% in moles, with respect to the polymer.

Preferably, the polymer with a sulfonic group is chosen from 2-acrylamido-2-methyl propane sulfonic acid homo- and co-polymers, salts thereof and mixtures thereof. Even more preferably, the polymer with a sulfonic group is chosen from 2-acrylamido-2-methyl propane sulfonic acid homopolymers, salts thereof and mixtures thereof.

The more particularly preferred 2-acrylamido 2-methyl propane sulfonic acid homopolymers comprise randomly distributed units with the following general formula (I):

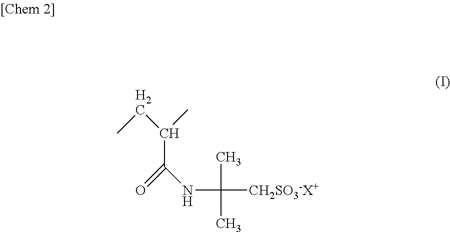

wherein X⁺ designates a proton, an alkaline metal cation, an alkaline-earth cation or the ammonium ion, at most 10% mol of X⁺ cations may consist of H⁺ protons;
and crosslinking agents originating from at least one monomer having at least two olefinic double bonds.

The homopolymers that are used, and more particularly preferred, according to the invention comprise from 90 to 99.9% by weight, and preferably from 98 to 99.5% by weight of units with the formula (I), and from 0.01 to 10% by weight, preferably from 0.2 to 2% by weight of crosslinking units, the weight proportions being defined with respect to the total weight of the polymer.

The preferred homopolymers of this type are chosen from crosslinked or non-crosslinked sodium 2-acrylamido-2-methyl propane sulfonate acid polymers such as that used in the commercial product SIMULGEL 800 (CTFA name: Sodium Polyacryloyldimethyl Taurate), or crosslinked polymers of ammonium 2-acrylamido-2-methyl propane sulfonate acid (INCI name: AMMONIUM POLYACRYLDIMEHYLTAURAMIDE) such as the product sold under the trade name HOSTACERIN AMPS® by Clariant.

The preferred copolymers of 2-acrylamido 2-methyl propane sulfonic acid are chosen from:
copolymers of 2-acrylamido-2-methylpropane sulfonic acid and acrylamide, for example of SEPIGEL or SIMULGEL type sold in particular by SEPPIC. By way of non-limiting examples of such copolymers, mention can for example be made of crosslinked anionic acrylamide/2-acrylamido 2-methyl propane sulfonic acid copolymers, in W/O emulsion form, such as those sold under the trade name Sepigel 305 (INCI name: polyacrylamide (and) C13-14 isoparaffin (and) laureth-7) and under the trade name Simulgel 600 (INCI name: acrylamide/sodium acryloyldimethyltaurate copolymer (and) isohexadecane (and) polysorbate 80) by SEPPIC;
copolymers of 2-acrylamido 2-methyl propane sulfonic acid and vinylpyrrolidone or vinylformamide such as that used in the commercial product sold under the trade name ARISTOFLEX AVC® by CLARIANT (CTFA name: AMMONIUM ACRYLOYLDIMETHYLTAURATE/VP COPOLYMER) but neutralized with soda or potash;
copolymers of 2-acrylamido 2-methyl propane sulfonic acid and sodium acrylate, such as for example the 2-acrylamido 2-methyl propane sulfonic acid/sodium acrylate copolymer such as that used in the commercial product sold under the trade name SIMULGEL EG® by SEPPIC or under the trade name SEPINOV EM (CTFA name: HYDROXYETHYL ACRYLATE/SODIUM ACRYLOYLDIMETHYL TAURATE COPOLYMER);
copolymers of 2-acrylamido 2-methyl propane sulfonic acid and hydroxyethyl acrylate, such as for example 2-acrylamido 2-methyl propane sulfonic/hydroxyethyl acrylate copolymer such as that used in the commercial product sold under the trade name SIMULGEL NS® by SEPPIC (CTFA name: HYDROXYETHYL ACRYLATE/SODIUM ACRYLOYLDIMETHYLTAURATE COPOLYMER (AND) SQUALANE (AND) POLYSORBATE 60) or as the product marketed under the name SODIUM ACRYLAMIDO-2-METHYLPROPANE SULFONATE/HYDROXYETHYLACRYLATE COPOLYMER such as the commercial product SEPINOV EMT 10 (INCI name: HYDROXYETHYL ACRYLATE/SODIUM ACRYLOYLDIMETHYL TAURATE COPOLYMER);
copolymers of 2-acrylamido 2-methyl propane sulfonic acid and ethoxylated cetearyl methacrylate, optionally crosslinked, such as Aristoflex® HMS, which bears the name 80/20 2-acrylamido 2-methyl propane sulfonic acid/ethoxylated (25 EO) cetearyl methacrylate copolymer, crosslinked with trimethylolpropane triacrylate (TMPTA) or Ammonium Acryloyldimethyltaurate/Steareth-25 methacrylate crosspolymer as INCI name;
and mixtures thereof.

Preferably, the polymer with a sulfonic group is a crosslinked homopolymer of ammonium 2-acrylamido-2-methyl propane sulfonate acid (INCI name: AMMONIUM POLYACRYLDIMEHYLTAURAMIDE) such as the product sold under the trade name HOSTACERIN AMPS® by Clariant.

Preferably, the polymer with a sulfonic group according to the invention is present in amounts of active substance ranging from 0.1 to 10% by weight, preferably from 0.5 to 5% by weight, more preferably from 0.7 to 2% by weight with respect to the total weight of the composition.

### Silicone Elastomer

The composition according to the invention comprises at least one silicone elastomer, also known as organopolysiloxane elastomer.

Preferably, the silicone elastomer is present in the oily phase, particularly silicone, of the O/W emulsion.

The expression “organopolysiloxane elastomer” denotes in the more generally definition thereof any chemically crosslinked siloxane polymer which has viscoelastic properties. Organopolysiloxane elastomers that can be used in the compositions according to the invention are particularly described in the application EP-A-0295886 and from those described in the U.S. Pat. Nos. 4,980,167 and 4,742,142, 5,266,321.

They are preferably non-emulsifying. The term “non-emulsifying” organopolysiloxane elastomers defines elastomers that do not contain any hydrophilic chains such as polyoxyalkylene or polyglycerolated units.

The silicone elastomer is a crosslinked organopolysiloxane elastomer that can be obtained by addition crosslinking reaction of organopolysiloxane containing at least one silicon-bonded hydrogen and organopolysiloxane having silicon-bonded ethylenic unsaturated groups, in particular in the presence of a platinum catalyst; or by condensation dehydrogenation crosslinking reaction between a hydroxyl-terminated diorganopolysiloxane and a diorganopolysiloxane that contains at least one silicon-bonded hydrogen, in particular in the presence of an organotin; or by condensation crosslinking reaction of a hydroxyl-terminated organopolysiloxane and a hydrolyzable organopolysilane; or by thermal crosslinking of organopolysiloxane, in particular in the presence of an organic peroxide catalyst; or by organopolysiloxane crosslinking by high-energy radiation such as gamma rays, ultraviolet rays, electron beams.

Organopolysiloxanes elastomers used in the composition according to the invention are preferably partially or totally crosslinked. They are presented in the form of particles. In particular, the organopolysiloxane elastomer particles have a size that can vary from 0.1 to 500 μm, preferably from 3 to 200 μm and even better from 3 to 50 μm. These particles can have any shape and for example be spherical, flat or amorphous.

The organopolysiloxane elastomer content in the compositions according to the invention can vary substantially, in particular according to the viscosity of the composition sought and according to the optional presence of an additional thickening agent. The optimal content according to these different parameters can be readily determined by a person skilled in the art using mere routine experiments.

As a general rule, the organopolysiloxane elastomer active substance content in the compositions according to the invention is from 0.1 to 5%, in particular from 0.5 to 3%, and more particularly from 1.0 to 2.5% by weight with respect to the total weight of the composition.

According to a specific embodiment, the organopolysiloxane elastomer is formulated in a vehicle comprising at least one non-volatile silicone oil. When they are included in this vehicle, the organopolysiloxane elastomers are transformed, according to the oil content used, into a product of spongy appearance when they are used in the presence of low oil contents, or into a homogeneous gel in the presence of a higher amount of oil. The gelling of the oil by these elastomers can be total or partial.

Such a mixture is known as “silicone agent”. Preferably, the organopolysiloxane elastomer is not formulated in a volatile silicone oil.

The term “oil” denotes any fatty substance in liquid form at ambient temperature (25° C.) and at atmospheric pressure (1.013·10⁵ Pa).

“Non-volatile oil” denotes, according to the invention, an oil having a non-zero vapor pressure and atmospheric pressure less than 0.13 Pa (10⁻³ mm of Hg).

Among the non-volatile silicone oils that can be used in the present invention, mention can be made for example of optionally phenylated non-volatile silicone oils, which advantageously have a molecular weight less than or equal to 150,000 g/mol, preferably less than or equal to 100,000 g/mol, and better less than or equal to 10,000 g/mol.

The expression “non-phenylated silicone oil” denotes a silicone oil that does not comprise any phenyl substituent. Examples that are representative of these non-phenylated non-volatile silicone oils that can be mentioned, comprise polydimethylsiloxanes (INCI name “dimethicone”), alkyldimethicones and vinylmethylmethicones.

Advantageously, these oils can be chosen from the following non-volatile oils:
polydimethylsiloxanes (PDMS),
alkyldimethicones comprising aliphatic groups, in particular alkyl, or alkoxy, which are pendant and/or at the end of the silicone chain; these groups each comprise from 2 to 24 carbon atoms. As an example mention can be made of cetyldimethicone sold under the trade name ABIL WAX 9801 from Evonik Goldschmidt,
polydimethylsiloxanes comprising functional groups such as hydroxyl groups,
substituted polydimethylsiloxane aliphatic groups, in particular C₂-C₂₄ alkyl, pendant and/or at the end of the silicone chain, and by functional groups such as hydroxyl groups, and
mixtures thereof.

Preferably, non-volatile linear polyorganosiloxane oils are used, particularly those having a viscosity less than or equal to 6 centistokes (6·10⁻⁶ m²/s).

Even more preferably, the non-volatile silicone oils are dimethicones.

According to a preferred embodiment of the invention, the silicone elastomer used in the preparation of the compositions according to the invention is supplied in the form of an organopolysiloxane elastomer as defined previously and formulated with active substance at a rate of 1 to 30%, and in particular from 10 to 20% by weight with respect to the total weight of said silicone agent (said silicone agent being the mixture of silicone elastomer and at least one silicone oil), in at least one non-volatile silicone oil as defined above.

The silicone agent defined above can act as a thickener in the compositions according to the invention. It can furthermore participate in the stabilization thereof.

Silicone elastomers such as those prepared by crosslinking addition reaction (A) of an organopolysiloxane having at least two silicon-bonded ethylenic unsaturated groups and (B) of a organopolysiloxane containing at least two hydrogen atoms each bonded to a different silicon atom, particularly in the presence (C) of platinum catalyst, as for example described in the application U.S. Pat. No. 4,980,167, are suitable for the compositions according to the invention.

In particular, the silicone elastomer can be obtained by reaction of dimethylvinylsiloxy-terminated dimethylpolysiloxane and of trimethylsiloxy-terminated methylhydrogenopolysiloxane, in the presence of a platinum catalyst.

The compound (A) is the basic reagent for the formation of silicone elastomer and the crosslinking takes place via addition reaction of the compound (A) with the compound (B) in the presence of the catalyst (C).

The compound (A) is advantageously an organopolysiloxane that has at least two lower alkenyl groups (for example in C2-C4); the lower alkenyl group can be chosen from the vinyl, allyl, and propenyl groups. These lower alkenyl groups can be located at any position of the organopolysiloxane molecule but are preferably located at the ends of the organopolysiloxane molecule. The organopolysiloxane (A) can have a branched chain, linear chain, cyclical or network structure but the linear chain structure is preferred. The compound (A) can have a viscosity ranging from the liquid state to the gum state. Preferably, the compound (A) has a viscosity of at least 100 centistokes at 25° C.

The organopolysiloxanes (A) may be chosen from methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl)polysiloxane, and dimethylvinylsiloxy-terminated dimethylsiloxane-methyl(3,3,3-trifluoropropyl)siloxane copolymers.

The compound (B) is in particular an organopolysiloxane that has at least 2 silicon-bonded hydrogens in each molecule and is therefore the cross-linker of the compound (A).

Advantageously, the sum of the number of ethylenic groups per molecule of the compound (A) and the number of silicon-bonded hydrogen atoms per molecule of the compound (B) is at least 4, preferably at least 5.

The compound (B) can be in any molecular structure, in particular a linear chain, branched chain, cyclical structure.

The compound (B) can have a viscosity at 25° C. ranging from 1 to 50,000 centistokes, in particular in order to be miscible with the compound (A).

It is advantageous that the compound (B) be added in an amount such that the molecular ratio between the total amount of all of the ethylenic unsaturated groups in the compound (A) and the total amount of silicon-bonded hydrogen atoms in the compound (B) be in the range from 1.5/1 to 20/1.

The compound (B) can be chosen from trimethylsiloxy-terminated methylhydrogenopolysiloxanes, trimethylsiloxy-terminated dimethylsiloxane-methylhydrogenosiloxane copolymers, dimethylsiloxane-methylhydrogenosiloxane cyclical polymers.

The compound (C) is the catalyst of the crosslinking reaction, and is in particular chloroplatinic acid, chloroplatinic-olefin acid complexes, chloroplatinic-alkenylsiloxane acid complexes, chloroplatinic-diketone acid complexes, black platinum, and supported platinum.

The catalyst (C) is preferably added from 0.1 to 1000 parts by weight, and better from 1 to 100 parts by weight, as a clean platinum metal for 1000 parts by weight of the total quantity of the compounds (A) and (B).

Other organic groups can be silicon bonded in the organopolysiloxane (A) and (B) described hereinabove, such as for example alkyl groups such as methyl, ethyl, propyl, butyl, octyl; substituted alkyl groups such as 2-phenylethyl, 2-phenylpropyl, 3,3,3-trifluoropropyl; alkyl groups such as phenyl, tolyl, xylyl; substituted aryl groups such as phenylethyl; and substituted monovalent hydrocarbon groups such as an epoxy group, an ester carboxylate group, a mercapto group.

Silicone polymers having an average molecular weight of at least 10,000 (for example ranging from 10,000 to 10,000,000) are also suitable as silicone elastomers according to the invention. Examples of silicone polymers include crosslinked siloxane copolymers, for example of dimethicone or dimethicone derivatives, such as stearyl methyl-dimethyl siloxane copolymer (“Gransil SR-CYC®” from Grant Industries), “Polysilicone-11®” (i.e., a crosslinked silicone elastomer formed by reacting silicone with a vinyl termination and methylhydrodimethyl siloxane), crosslinked cetearyl dimethicone/vinyl dimethicone copolymers (i.e., a crosslinked cetearyl dimethicone copolymer with a vinyl dimethyl polysiloxane), a crosslinked dimethicone/phenyl vinyl dimethicone copolymer (i.e., a crosslinked dimethylpolysiloxane copolymer with phenyl vinyl dimethylsiloxane) and a crosslinked dimethicone/vinyl dimethicone copolymer (i.e., a crosslinked dimethylpolysiloxane copolymer with vinyl dimethylsiloxane).

Such silicone gels can be obtained commercially particularly from Grant Industries. Examples of such gels comprise the mixtures of dimethicone and polysilicone-11 for example sold under the trade name “Gransil DMG-6/LC®”.

Further silicone gels can also be obtained commercially particularly from Shin-Etsu under the following reference: KSG-16.

### Filler

The composition according to the invention can comprise at least one filler.

The filler can be inorganic or organic.

The mineral fillers can be chosen from synthetic or natural mica; silica powder; talc; kaolin; boron nitride or mixtures thereof.

The organic fillers can be chosen from:
organopolysiloxane powders coated with silicone resin, such as those sold under the trade names “KSP-100”, “KSP-101”, “KSP-102”, “KSP-103”, KSP-104”, “KSP-105” by Shin Etsu,
polymethylsilsesquioxane powders, such as those sold under the trade name TOSPEARL by Momentive Performance Materials;
polyamide powders also known as Nylon such as Nylon-1 (Polyamide 1), Nylon-12 (Polyamide 12), such as those sold under the trade names ORGASOL by Arkema Nylon-66 (Polyamide 66); Nylon-6 (Polyamide 6);
polyethylene powders;
microspheres based on acrylic copolymers, such as those made of ethylene glycol dimethacrylate/lauryl methacrylate copolymer sold by Dow Corning under the trade name POLYTRAP;
expanded powders such as hollow microspheres and in particular, microspheres sold under the trade name EXPANCEL by Nouryon;
methyl polymethacrylarte microspheres, such as those sold under the trade name MICROSPHERE M-100 by Matsumoto or under the trade name COVABEAD LH85 by Sensient;
ethylene-acrylate copolymer powders, such as those sold under the trade name FLOBEADS by Sumitomo Seika Chemicals;
natural organic material powders such as starch powders particularly corn, wheat or rice starches, optionally crosslinked, such as starch powders crosslinked with octenylsuccinate anhydride, such as those marketed under the trade name DRY-FLO by Nouryon;
poly-p-phenylene terephtamide powders;
and mixtures thereof.

Preferably, the composition according to the invention can comprise at least one mineral filler such as a silica powder.

Alternatively, the composition according to the invention comprises at least one silicone filler.

Preferably, the silicone filler is chosen from:
organopolysiloxane powders coated with silicone resin;
polymethylsilsesquioxane powders; and
mixtures thereof.

According to a first advantageous embodiment of the invention, the composition comprises by way of silicone filler, an organopolysiloxane powder coated with silicone resin. According to this embodiment, the organopolysiloxane powder can particularly be coated with silsesquioxane resin, as described for example in the U.S. Pat. No. 5,538,793. Such elastomer powders are sold under the trade names “KSP-100”, “KSP-101”, “KSP-102”, “KSP-103”, KSP-104”, “KSP-105” by Shin Etsu, and have the INCI name: vinyl dimethicone/methicone silsesquioxane crosspolymer.

According to a second advantageous embodiment of the invention, the composition can comprise at least one polymethylsilsesquioxane powder, in particular silicone resin microbeads such as those sold under the trade name TOSPEARL by Momentive Performance Materials, and particularly under the reference Tospearl 145 A, and mixtures thereof.

Advantageously, the filler is chosen from synthetic or natural mica; silica powder; talc; and mixtures thereof.

In particular, the composition according to the invention can comprise a filler at a content ranging from 0.5% to 20% by weight, with respect to the total weight of the composition, particularly from 0.7% to 15% by weight, preferably ranging from 1% to 10% by weight, and preferably ranging from 1% to 5% by weight.

### pH of the Composition

The composition according to the invention has a pH between 4.5 and 6.5. Advantageously, the pH of the composition is between 5.0 and 6.0.

Preferably, the cosmetic composition according to the invention comprises at least one base.

The base is particularly used to increase the final pH of the composition to between 4.5 and 6.5, preferably between 5.0 and 6.0.

The base can be chosen from mineral bases such as for example alkali metal hydroxides, sodium hydroxide, potassium hydroxide.

Preferably, the base of the composition is an alkali metal hydroxide, preferably sodium hydroxide or potassium hydroxide. Indeed, these bases are more advantageous than nitrogenous bases, such as triethanolamine, on color stabilization (i.e., less yellowing).

The composition according to the invention preferably comprises at least one base at an active substance content ranging from 0.5% to 10% by weight, with respect to the total weight of the composition, particularly from 1% to 5% by weight, preferably ranging from 1% to 4% by weight.

### Aqueous Phase

Besides ascorbic acid or one of the derivatives thereof, the aqueous phase of the O/W emulsion according to the invention comprises a physiologically acceptable aqueous medium. Said medium comprises water.

The water used can be sterile demineralized water and/or floral water such as rose water, cornflower water, chamomile water or linden water, and/or a spring or natural mineral water.

The composition preferably comprises at least 30% by weight of water with respect to the total weight of the composition, preferably at least 40% by weight.

The composition preferably comprises from 30% to 85% by weight of water with respect to the total weight of the composition, more preferably from 40% to 80%, even more preferably from 45% to 70%.

The aqueous phase can also comprise at least one organic solvent miscible in water, at 25° C.

Preferably, the water-miscible organic solvent is chosen from alcohols, polyols and mixtures thereof.

Among the alcohols, mention can be made of C₁-C₁₀, more preferably C₁-C₅, alcohols, such as ethanol, isopropanol, propanol and butanol.

The polyol is, preferably, chosen from polyols having from 2 to 20 carbon atoms, preferably from 2 to 6 carbon atoms, such as glycerol, diglycerol, propyleneglycol, isoprene glycol, dipropyleneglycol, butylene glycol, hexylene glycol, 1,2-propanediol, 1,3-propanediol, pentylene glycol, polyethyleneglycols having from 2 to 200 ethylene oxide units and mixtures thereof.

Preferably, the composition comprises from 1% to 25% by weight of water-miscible organic solvent, with respect to the total weight of the composition, more preferably from 2% to 20% by weight, even more preferably from 5% to 15% by weight.

### Oily Phase

Besides silicone compounds, the composition according to the invention comprises at least one oily phase, which preferably comprises at least one oil, particularly a cosmetic oil. It can further contain other fats.

The oily phase is therefore also the silicone phase.

By way of oil suitable for the implementation of the invention, mention can particularly be made of:
hydrocarbon oils of animal origin,
hydrocarbon oils of plant origin, such as phytostearyl esters, such as phytostearyl oleate, phytostearyl isostearate and lauroyl/octyldodecyl/phytostearyl glutamate, for example sold under the trade name ELDEW PS203® by AJINOMOTO, triglycerides constituted of esters of fatty acids and glycerol for which the fatty acids can have chain lengths ranging from C₄ to C₂₄, with the latter able to be linear or branched, saturated or unsaturated; these oils are particularly heptanoic or octanoic oils, wheat germ, sunflower, grape seed, sesame, corn, apricot, castor, camelina, shea, avocado, olive, soybean, sweet almond, palm, rapeseed, cotton, hazelnut, macadamia, jojoba, alfalfa, poppy seed, pumpkin, squash, blackcurrant, evening primrose, millet, barley, quinoa, rye, safflower, candlenut, passiflora, musk rose oils; or caprylic/capric acid triglycerides such as those sold by STEARINERIES DUBOIS or those sold under the trade names MIGLYOL 810®, 812® and 818® by DYNAMIT NOBEL, refined plant perhydrosqualene sold under the trade name Fitoderm by Cognis,
synthetic esters such as oils of formula R₁COOR₂ wherein R₁ represents the residue of a linear or branched fatty acid including 1 to 40 carbon atoms and R₂ represents a particularly branched hydrocarbon chain containing 1 to 40 carbon atoms on the condition that the sum of R₁ and R₂ is greater than or equal to 10. The esters can be particularly chosen from esters, particularly of fatty acid such as for example cetostearyl octanoate, isopropyl alcohol and C8-C18, preferably C12-C16, fatty acid esters such as isopropyl palmitate, ethyl palmitate, 2-ethyl-hexyl palmitate, isopropyl myristate, isopropyl stearate or isostearate, isostearyl isostearate, octyl stearate, hydroxylated esters such as isostearyl lactate, octyl hydroxystearate, diisopropyl adipate, heptanoates, and particularly isostearyl heptanoate, octanoates, decanoates or ricinoleates of alcohols or polyalcohols such as propylene glycol dioctanoate, cetyl octanoate, tridecyl octanoate, 2-ethylhexyl 4-diheptanoate and palmitate, alkyl benzoate, polyethylene glycol diheptanoate, propylene glycol diethyl 2-hexanoate and mixtures thereof, C₁₂ to C₁₅ alcohol benzoates, hexyl laurate, neopentanoic acid esters such as isodecyl neopentanoate, isotridecyl neopentanoate, isostearyl neopentanoate, octyldodecyl neopentanoate, isononanoic acid esters such as isononyl isononanoate, isotridecyl isononanoate, octyl isononanoate, hydroxylated esters such as isostearyl lactate, di-isostearyl malate;
polyol esters, and pentaerythritol esters, such as pentaerythrityl tetraoctanoate or dipentaerythrityl tetrahydroxystearate/tetraisostearate,
mixtures thereof.

Preferably, the composition comprises from 1% to 25% by weight of oily phase, particularly silicone, with respect to the total weight of the composition, more preferably from 2% to 20% by weight, even more preferably from 5% to 15% by weight.

The compositions of the invention can contain one or more of the usual active agents or excipients in the cosmetic and dermatological fields, such as hydrating agents; emollients; gelling agents, for example natural gelling agents; active agents; antioxidants; perfumes; film-forming agents; dyestuffs; and mixtures thereof. The amounts of these different excipients are those conventionally used in the fields in question. In particular, these amounts vary according to the sought purpose and can for example range from 0.01% to 20%, and preferably from 0.1% to 10% by weight of the total weight of the composition. Obviously, those skilled in the art will take care to choose the optional active agent(s) or excipient(s) added to the composition according to the invention in such a way that the advantageous properties intrinsically associated with the composition according to the invention are not altered, or are not substantially altered, by the envisaged addition.

The composition according to the invention is substantially free of cyclohexasiloxane. Substantially free of cyclohexasiloxane means that the composition comprises less than 1% by weight, preferably less than 0.5% by weight, preferably less than 0.3% by weight, preferably less than 0.1% by weight with respect to the total composition weight of the composition of cyclohexasiloxane. Preferably, the composition according to the invention is totally free of cyclohexasiloxane.

Preferably, the composition according to the invention is substantially free of volatile silicone. Besides cyclohexasiloxane volatile silicone oil, mention can also be made of cyclotetrasiloxane, cyclopentasiloxane silicone oils. Substantially free of volatile silicone means that the composition comprises less than 0.5% by weight, preferably less than 0.3% by weight, preferably less than 0.1% by weight with respect to the total weight of the composition of volatile silicone. Preferably, the composition according to the invention is totally free of volatile silicone.

Preferably, the composition according to the invention is substantially free of preservative. The preservative is for example 2,6-di-tert-butyl 4-methylphenol (or butylhydroxytoluene or BHT), a paraben or benzyl alcohol. Substantially free of preservative means that the composition comprises less than 0.3% by weight, preferably less than 0.1% by weight with respect to the total weight of the composition of preservative. Preferably, the composition according to the invention is totally free of preservative.

Preferably, the composition according to the invention is substantially free of chelating agent. The chelating agent is for example EDTA. Substantially free of chelating agent means that the composition comprises less than 0.1% by weight, preferably less than 0.05% by weight with respect to the total weight of the composition of chelating agent. Preferably, the composition according to the invention is totally free of chelating agent.

Preferably, the composition according to the invention is substantially free of volatile silicone, preservative and chelating agent.

### Viscosity

Preferably, the composition according to the invention has a fluid viscosity specific to a serum, which enables the packaging and delivery of the composition in a pipette bottle. Preferably, the viscosity at 25° C. is between 0.1 and 1 Pa·s, more preferably between 0.4 and 0.9 Pa·s, more preferably between 0.5 and 0.8 Pa·s.

The measurement protocol of the viscosity is as follows (protocol A):

The viscosity is measured with a Rheomat RM200 apparatus equipped with the spindle M3.

The measures are made at a temperature of 25° C.+/−0.5° C. after 10 minutes of rotation of the spindle at a speed of 200 rpm.

The invention also relates to the cosmetic use of a composition according to the invention for the care of keratin materials, in particular for body and/or facial skin care.

The present invention further relates to a non-therapeutic cosmetic care method of keratin materials such as the skin, in particular body and/or facial skin, wherein a composition according to the invention is applied on said keratin materials.

The composition according to the invention can be obtained conventionally by those skilled in the art.

The following examples make it possible to understand the invention better, without being in any way limitative. The raw materials are referred to with the chemical or INCI name thereof. The amounts indicated are as a % by weight of raw materials with respect to the total weight of the composition (% w/w), unless specified otherwise.

### EXAMPLE 1: PREPARATION OF COMPOSITIONS ACCORDING TO THE INVENTION AND A COMPARATIVE COMPOSITION

The compositions C1 and C2 according to the invention, and comparative composition CC* (marked by an asterisk), were prepared according to the following method:
Phase A1 under heated stirring at 65° C.;
Phase E mixed using a magnetic stirrer at ambient temperature until complete solubilization of the ascorbic acid. Exothermic phase prepared in an iced water-bath;
Phase F1 under magnetic stirring until complete solubilization of the salicylic acid and the active agent;
Phase F2 under Rayneri type stirring using a deflocculator type shaft under the mixture is completely smooth.

### Implementation of the Formula Under MORITZ TURBOLAB 2400 Type Stirrer:

At an increasing stirring speed (rpm) proportional to the viscosity of the mixture:
Introduction of dilution water (i.e. phase A2) in phase A1;
Cooling;
Then introduction of phase B into phase A for the gelling of the formula;
Disperse until a smooth grain-free gel is obtained;
Introduction of phases C and D;
Disperse until a homogeneous phase is obtained;
Introduction of phase E;
Introduction of phase F1—Disperse until a homogeneous phase is obtained—Introduction of phase F2.

The composition CC* is comparative as it contains no silicone emulsifier according to the invention.

TABLE 1

| | Compo- | Compo- | Compo- | |
|---|---|---|---|---|
| | sition | sition | sition | |
| INCI name | C1% w/w | C2% w/w | CC* % w/w | Phase |
| Water | Qs 100 | Qs 100 | Qs 100 | A1 |
| (of which a fraction of La | | | | |
| Roche Posay thermal spring | | | | |
| water) | | | | |
| Glycerin | 7 | 7 | 7 | A1 |
| Adenosine | 0.1 | 0.1 | 0.1 | A1 |
| PEG-20 METHYL | 1 | 1 | 0.5 | A1 |
| GLUCOSE | | | | |
| SESQUISTEARATE | | | | |
| (GLUCAMATE SSE-20 | | | | |
| EMULSIFIER by | | | | |
| LUBRIZOL) | | | | |
| Poloxamer 338 | 0.6 | 0.6 | 0.5 | A1 |
| (SYNPERONIC PE/F 108- | | | | |
| NAA-FL-(CQ) by | | | | |
| CRODA) | | | | |
| Caprylyl glycol | 0.3 | 0.3 | 0.3 | A1 |
| Disodium EDTA | — | — | 0.1 | A2 |
| Water | 22 | 20.6 | 22 | A2 |
| Xanthan gum | 0.2 | 0.2 | 0.1 | B |
| AMMONIUM | 0.8 | 0.7 | 0.7 | B |
| POLYACRYLOYLDI- | | | | |
| METHYL TAURATE | | | | |
| (Hostacerin AMPS ® by | | | | |
| Clariant) | | | | |
| Sodium hyaluronate | 0.3 | 0.3 | 0.3 | B |
| Hydrolyzed hyaluronic acid | 0.1 | 0.1 | 0.1 | B |
| POLYACRYLAMIDE | 0.8 | 0.8 | 0.8 | C |
| (and) C13-14 | | | | |
| ISOPARAFFIN (and) | | | | |
| LAURETH-7 | | | | |
| (Sepigel 305 by SEPPIC) | | | | |
| PENTAERYTHRITYL | 1 | 1 | 1 | D |
| TETRAETHYL- | | | | |
| HEXANOATE | | | | |
| Ascorbic acid | 10 | 10 | 10 | E |
| Water | 10 | 10 | 10 | E |
| Potassium hydroxide in | 5.9 | 5.9 | 5.9 | E |
| aqueous solution (50% | (2.95 a.s.) | (2.95 a.s.) | (2.95 a.s.) | |
| active substance or a.s.) | | | | |
| Ethanol | 5 | 5 | 5 | F1 |
| Salicylic acid | 0.45 | 0.45 | 0.45 | F1 |
| Active agent | Qs | Qs | Qs | F1 |
| DIMETHICONE (and) | 9 | 10 | 10 | F2 |
| POLYSILICONE-11 | | | | |
| (Gransil DMG-6/LC ® by | | | | |
| Grant Industries) | | | | |
| BIS-PEG/PPG-16/16 | 0.5 | 0.5 | — | F2 |
| PEG/PPG-16/16 | | | | |
| DIMETHICONE (and) | | | | |
| CAPRYLIC/CAPRIC | | | | |
| TRIGLYCERIDE | | | | |
| (Abil Care 85 by | | | | |
| Goldschmidt) | | | | |
| Dimethicone (viscosity 100 | 1 | 1 | 1 | F2 |
| cSt) | | | | |
| Polymethylsilsesquioxane | 1.5 | — | 1.5 | F2 |
| (TOSPEARL 2000 B | | | | |
| MICROSPHERE by | | | | |
| MOMENTIVE | | | | |
| PERFORMANCE | | | | |
| MATERIALS) | | | | |
| Silica | — | 2 | — | F2 |
| (BA4 by JGC | | | | |
| CATALYSTS & | | | | |
| CHEMICALS) | | | | |
| Perfume | 0.3 | 0.4 | 0.5 | F2 |

The stability at 2 months of compositions C1, C2 and CC* was assessed, at ambient temperature (AT), at 4° C., at 37° C. and at 45° C.

For this, the appearance, color and odor of the compositions were assessed after 2 months under each condition.

The appearance is considered to be compliant if the composition has no granular texture, and non-compliant otherwise.

Color and odor: the color is considered to be compliant when it is white to very slightly yellow, and non-compliant if stronger yellowing is observed. The odor is considered to be compliant when the odor provided by the perfume has little or no modification.

Moreover, at AT and at 45° C., the pH and the viscosity of the compositions were measured. The viscosity measurement protocol is protocol A described hereinabove in the description.

Finally, microscopic images (×10, with the Leica DM2500 optical microscope) in natural light were taken for each composition, after 2 months at ambient temperature (TA), at 4° C., at 37° C. and at 45° C.

The results are as follows:

### C1:

### 2-Month Stability:

Appearance: fluid translucent emulsion
Color & odor: very pale yellow, fragranced
AT: pH=5.4 and viscosity between 0.69 and 0.73 Pa·s
45° C.: pH=5.9 and viscosity between 0.57 and 0.6 Pa·s
×10 microscopy in natural light at T2 months: AT and 45° C.: the emulsion is thin and the edges are clearly defined. The formula is irregular anisotropic.

### C2:

### 2-Month Stability:

Appearance: fluid translucent emulsion
Color & odor: very pale yellow, fragranced
AT: pH=5.4 and viscosity between 0.73 and 0.82 Pa·s
45° C.: pH=5.8 and viscosity between 0.51 and 0.57 Pa·s
×10 microscopy in natural light at T2 months: AT and 45° C.: the emulsion is thin and the edges are clearly defined. The formula is irregular anisotropic.

### CC*:

### 2-Month Stability:

Appearance: granular texture, non-compliant at AT, 4° C., 37° C. and 45° C.
Color & odor: compliant at 4° C. and modified at AT, 37° C. and 45° C. (i.e., vitamin C oxidation).
AT: pH=5.5 and non-compliant viscosity of 0 Pa·s.
45° C.: pH=5.8 and non-compliant viscosity of 0 Pa·s.
×10 microscopy in natural light at T2 months: non-compliant degraded.

It is apparent from these results that only compositions C1 and C2 according to the invention are stable under the various test conditions.

### EXAMPLE 2: PREPARATION OF A COMPOSITION ACCORDING TO THE INVENTION

Composition 03 according to the invention were prepared according to the protocol in example 1.

TABLE 2

| | Composition | |
|---|---|---|
| INCI name | C3% w/w | Phase |
| Water | Qs 100 | A1 |
| (of which a fraction of La Roche Posay thermal | | |
| spring water) | | |
| Glycerin | 7 | A1 |
| Adenosine | 0.1 | A1 |
| PEG-20 METHYL GLUCOSE | 1 | A1 |
| SESQUISTEARATE | | |
| (GLUCAMATE SSE-20 EMULSIFIER by | | |
| LUBRIZOL) | | |
| Poloxamer 338 | 0.6 | A1 |
| (SYNPERONIC PE/F 108-NAA-FL-(CQ) by | | |
| CRODA) | | |
| Caprylyl glycol | 0.3 | A1 |
| Water | 22 | A2 |
| Xanthan gum | 0.15 | B |
| AMMONIUM | 0.6 | B |
| POLYACRYLOYLDIMETHYL TAURATE | | |
| (Hostacerin AMPS ® by Clariant) | | |
| Sodium hyaluronate | 0.3 | B |
| Hydrolyzed hyaluronic acid | 0.1 | B |
| POLYACRYLAMIDE (and) C13-14 | 0.7 | C |
| ISOPARAFFIN (and) LAURETH-7 | | |
| (Sepigel 305 by SEPPIC) | | |
| PENTAERYTHRITYL | 1 | D |
| TETRAETHYLHEXANOATE | | |
| Ascorbic acid | 10 | E |
| Water | 10 | E |
| Potassium hydroxide in aqueous solution (50% | 5.9 | E |
| active substance or a.s.) | (2.95 a.s.) | |
| Ethanol | 5 | F1 |
| Salicylic acid | 0.45 | F1 |
| Active agent | Qs | F1 |
| DIMETHICONE (and) POLYSILICONE-11 | 9 | F2 |
| (Gransil DMG-6/LC ® by Grant Industries) | | |
| BIS-PEG/PPG-16/16 PEG/PPG-16/16 | 0.5 | F2 |
| DIMETHICONE (and) CAPRYLIC/CAPRIC | | |
| TRIGLYCERIDE | | |
| (Abil Care 85 by Goldschmidt) | | |
| Dimethicone (viscosity 100 cSt) | 1 | F2 |
| Silica | 1.5 | F2 |
| (SOLESPHERE H 51 by AGC SI-TECH) | | |
| Perfume | 0.4 | F2 |

1. A composition in the form of an oil-in-water emulsion having a pH of between 4.5 and 6.5, comprising:
a) at least 5% by weight of ascorbic acid or one of the derivatives thereof with respect to the total weight of the composition;
b) at least one silicone emulsifier;
c) at least one polymer with a sulfonic group;
d) at least one silicone elastomer; and
e) optionally at least one filler;
said composition being substantially free of cyclohexasiloxane.

2. The composition according to claim 1, characterized in that the ascorbic acid derivative is chosen from 5,6-di-O-dimethylsilylascorbate, DL-alpha-tocopheryl-DL-ascorbyl-phosphate potassium salt, magnesium ascorbyl phosphate, sodium ascorbyl phosphate, disodium ascorbyl sulfate, sulfinyl-L-ascorbic acid, glucopyranosyl-L-ascorbic acid and ascorbyl glucoside.

3. The composition according to claim 1, characterized in that it comprises at least 7% by weight of ascorbic acid or one of the derivatives thereof with respect to the total weight of the composition.

4. The composition according to claim 1, characterized in that the silicone emulsifier is chosen from dimethicone copolyols.

5. The composition according to claim 1, characterized in that the silicone emulsifier is a dimethicone copolyol.

6. The composition according to claim 1, characterized in that the silicone emulsifier is present at an active substance content ranging from 0.1 to 5% by weight with respect to the total weight of the composition.

7. The composition according to claim 1, characterized in that the polymer with a sulfonic polymer is a poly (2-acrylamido 2-methyl propane sulfonic) acid polymer.

8. The composition according to claim 1, characterized in that the polymer with a sulfonic group is chosen from crosslinked or non-crosslinked sodium 2-acrylamido-2-methyl propane sulfonate homopolymers, crosslinked ammonium 2-acrylamido-2-methyl propane sulfonate homopolymers, 2-acrylamido-2-methylpropane sulfonic acid and acrylamide copolymers, 2-acrylamido-2-methyl propane sulfonic acid and vinylpyrrolidone or vinylformamide copolymers, 2-acrylamido-2-methyl propane sulfonic acid and sodium acrylate copolymers, 2-acrylamido-2-methyl propane sulfonic acid and hydroxyethyl acrylate copolymers, 2-acrylamido-2-methyl propane sulfonic acid and ethoxylated cetearyl methacrylate copolymers, optionally crosslinked, and mixtures thereof.

9. The composition according to claim 1, characterized in that the polymer with a sulfonic group is present in active substance amounts ranging from 0.1 to 10% by weight with respect to the total weight of the composition.

10. The composition according to claim 1, characterized in that the silicone elastomer is present at an active substance content ranging from 0.1 to 5% with respect to the total weight of the composition.

11. The composition according to claim 1, characterized in that the silicone elastomer is formulated in a vehicle comprising at least one non-volatile silicone oil.

12. The composition according to claim 1, characterized in that the silicone elastomer is prepared by crosslinking reaction between polysiloxanes (A) containing groups ≡Si—H, an alpha,omega-diene (B) in the presence of a catalyst, and a low molecular weight linear or cyclic polysiloxane (C).

13. The composition according to claim 1, characterized in that it has a pH of between 5.0 and 6.0.

14. The composition according to claim 1, characterized in that it comprises less than 1% by weight with respect to the total composition weight of the composition of cyclohexasiloxane.

15. The composition according to claim 1, characterized in that it comprises an aqueous phase comprising water and optionally at least one water-soluble organic solvent, at 25° C.

16. The composition according to claim 1, characterized in that it comprises a mineral or organic filler.

17. (canceled)

18. The non-therapeutic cosmetic care method for keratin materials, wherein a composition according to claim 1 is applied on said keratin materials.

19. The composition according to claim 2, characterized in that it comprises at least 7% by weight of ascorbic acid or one of the derivatives thereof with respect to the total weight of the composition.

20. The composition according to claim 2, characterized in that the silicone emulsifier is chosen from dimethicone copolyols.

21. The composition according to claim 3, characterized in that the silicone emulsifier is chosen from dimethicone copolyols.
